(19) United States
(12) Patent Application Publication
(10) Pub. No.: US 2023/0105978 A1
(43) Pub. Date: Apr. 6, 2023

(54) DOUBLE-LAYERED MEDICAL MEMBRANE AND METHOD FOR MANUFACTURING THE SAME

(71) Applicant: NAN YA PLASTICS CORPORATION, TAIPEI (TW)

(72) Inventors: TE-CHAO LIAO, TAIPEI (TW); Min-Fan Chung, Taoyuan City (TW); CHING-YAO YUAN, TAIPEI (TW); WEN-JUI CHENG, TAIPEI (TW)

(21) Appl. No.: 17/855,702

(22) Filed: Jun. 30, 2022

(30) Foreign Application Priority Data
Oct. 1, 2021 (TW) 110136633

(51) Int. Cl. A61L 15/22; A61L 15/60

(52) U.S. Cl. CPC A61L 15/22; A61L 15/60

(57) ABSTRACT

A double-layered medical membrane and a method for manufacturing the same are provided. The double-layered medical membrane includes a hydrophilic material layer and a hydrophobic material layer disposed thereon. The hydrophilic material layer includes hydrophilic fibers, hydrophilic particles, or a combination thereof. The hydrophilic fibers and the hydrophilic particles include a hydrophilic polymer as a material. The method includes the following steps: using an electrospinning solution to form the hydrophilic material layer by electrospinning or using a hydrophilic solution to form the hydrophilic material layer; and producing the double-layered medical membrane that includes the hydrophilic material layer. The hydrophilic material layer includes the hydrophilic fibers, the hydrophilic particles, or the combination thereof. The hydrophilic fibers or the hydrophilic particles include the hydrophilic polymer as a material.

### CROSS-REFERENCE TO RELATED PATENT APPLICATION

This application claims the benefit of priority to Taiwan Patent Application No. 110136633, filed on Oct. 1, 2021. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a double-layered medical membrane and a method for manufacturing the same, and more particularly to a double-layered medical membrane that has an anti-adhesive effect and a method for manufacturing the same.

### BACKGROUND OF THE DISCLOSURE

Various wounds can be formed inside the body of a patient after a surgery is performed. During a healing process, body fluids or blood may seep out of the wound, such that adhesion is likely to occur once the wound comes in contact with other organs. To prevent occurrences of the adhesion, a doctor will cover the wound with an anti-adhesion membrane after the surgery, so that chances of the adhesion occurring can be reduced.

Conventional anti-adhesion membranes are all formed in a single-layer structure, and a component by which the anti-adhesion membrane is generally formed is polylactic acid or carboxymethyl cellulose. However, such an anti-adhesion membrane (whether being formed by the polylactic acid or the carboxymethyl cellulose) still has certain deficiencies and is inconvenient for use.

The polylactic acid is of a hydrophobic material. As such, the anti-adhesion membrane formed by the polylactic acid has a problem of not easily adhering to the wound, so that a suture operation still needs to be performed to fix the anti-adhesion membrane to the wound.

The anti-adhesion membrane formed by the carboxymethyl cellulose can more easily attach to the wound. However, the carboxymethyl cellulose has a tendency to swell after absorption of water, and is thus likely to adhere to medical gloves or devices. Therefore, a TYVEK® paper is usually used in cooperation with the anti-adhesion membrane formed by the carboxymethyl cellulose. Having the anti-adhesion membrane covered by the TYVEK® paper can make the suture operation or other treatments to be performed more convenient for the doctor.

According to the above, a user of the conventional anti-adhesion membrane (whether being formed by the polylactic acid or the carboxymethyl cellulose) can still experience a number of issues during use, so that there is still room for improvement in the conventional anti-adhesion membrane.

### SUMMARY OF THE DISCLOSURE

In response to the above-referenced technical inadequacies, the present disclosure provides a double-layered medical membrane and a method for manufacturing the same.

In one aspect, the present disclosure provides a double-layered medical membrane. The double-layered medical membrane includes a hydrophilic material layer and a hydrophobic material layer. The hydrophilic material layer includes hydrophilic fibers, hydrophilic particles, or a combination thereof. The hydrophilic fibers and the hydrophilic particles include a hydrophilic polymer as a material. The hydrophobic material layer is disposed on the hydrophilic material layer.

In certain embodiments, the hydrophilic polymer is selected from the group consisting of hyaluronic acid, carboxymethyl cellulose, collagen, gelatin, alginate, and chitosan.

In certain embodiments, a molecular weight of the hydrophilic polymer is from 2,000 to 2,000,000.

In certain embodiments, the hydrophilic fibers further include an excipient as a material, and a weight ratio between the hydrophilic polymer of the hydrophilic fibers and the excipient is from 1:30 to 5:1.

In certain embodiments, the hydrophobic material layer is formed by hydrophobic fibers, and the hydrophobic fibers have a diameter that is from 200 nm to 3,000 nm.

In certain embodiments, the hydrophobic material layer is formed by electrospinning.

In certain embodiments, a tensile strength of the double-layered medical membrane is from 1.1 MPa to 5.2 Mpa.

In another aspect, the present disclosure provides a method for manufacturing a double-layered medical membrane. The method includes: using an electrospinning solution to form a hydrophilic material layer by electrospinning; and producing the double-layered medical membrane that includes the hydrophilic material layer. The hydrophilic material layer includes hydrophilic fibers, and the hydrophilic fibers include a hydrophilic polymer as a material. The double-layered medical membrane further includes a hydrophobic material layer that is disposed on the hydrophilic material layer.

In certain embodiments, the hydrophilic fibers further include an excipient as a material. The electrospinning solution includes: 5 wt % to 75 wt % of alcohol, 25 wt % to 95 wt % of water, 0.5 wt % to 5 wt % of the hydrophilic polymer, and 2 wt % to 15 wt % of the excipient.

In yet another aspect, the present disclosure provides a method for manufacturing a double-layered medical membrane. The method includes: using a hydrophilic solution to form a hydrophilic material layer; and producing the double-layered medical membrane that includes the hydrophilic material layer. The hydrophilic material layer includes hydrophilic fibers, hydrophilic particles, or a combination thereof. The hydrophilic fibers and the hydrophilic particles include a hydrophilic polymer as a material. The double-layered medical membrane further includes a hydrophobic material layer that is disposed on the hydrophilic material layer.

Therefore, in the double-layered medical membrane and the method for manufacturing the same provided by the present disclosure, by virtue of “the hydrophilic material layer being formed by the hydrophilic fibers, the hydrophilic particles, or the combination thereof” and “the hydrophobic material layer being disposed on the hydrophilic material layer,” inconveniences associated with using a conventional single-layered medical membrane can be solved.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view showing a double-layered medical membrane according to the present disclosure in use;

FIG. 2 is a schematic cross-sectional view of the double-layered medical membrane according to the present disclosure;

FIG. 3 is an enlarged view of part III of FIG. 2;

FIG. 4 is a schematic view of an electrospinning device configured to implement a method for manufacturing the double-layered medical membrane according to the present disclosure;

FIG. 5 is an enlarged view of part V of FIG. 2;

FIG. 6 is a flowchart of the method for manufacturing the double-layered medical membrane according to one embodiment of the present disclosure;

FIG. 7 is a flowchart of the method for manufacturing the double-layered medical membrane according to another embodiment of the present disclosure; and

FIG. 8 is a flowchart of the method for manufacturing the double-layered medical membrane according to yet another embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

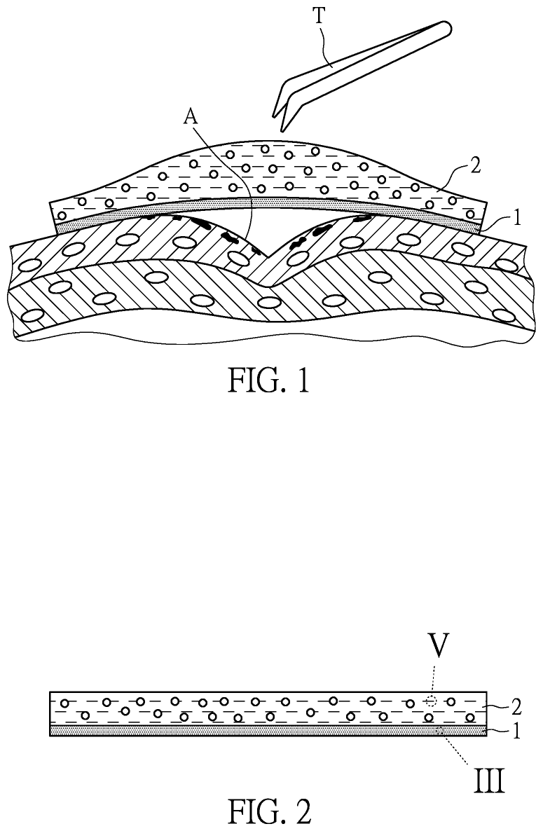

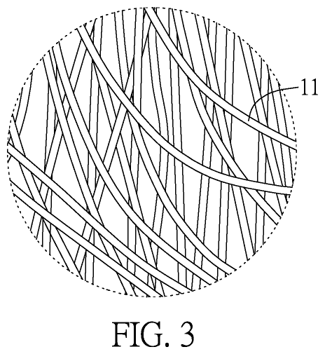

Referring to FIG. 1 and FIG. 2, a double-layered medical membrane of the present disclosure includes a hydrophilic material layer 1 and a hydrophobic material layer 2 that are arranged in a stacked manner. The hydrophilic material layer 1 and the hydrophobic material layer 2 are directly joined with each other. The hydrophilic material layer 1 can be in contact with a wound A, and has good adhesion ability. The hydrophobic material layer 2 can prevent the wound A from having an adhesion with surrounding organs, and does not easily adhere to a medical device T or medical consumables. Accordingly, the double-layered medical membrane of the present disclosure has improved convenience of use.

A total thickness of the double-layered medical membrane is from 10 μm to 100 μm, so as to allow for convenient use. A thickness of the hydrophilic material layer 1 is from 5 μm to 50 μm, and a thickness of the hydrophobic material layer 2 is from 5 μm to 90 μm. Preferably, the thickness of the hydrophobic material layer 2 is greater than the thickness of the hydrophilic material layer 1. More preferably, a thickness ratio between the hydrophobic material layer 2 and the hydrophilic material layer 1 is from 2:1 to 6:1. In this way, the double-layered medical membrane of the present disclosure can be conveniently held.

The hydrophilic material layer 1 of the present disclosure is formed by electrospinning or coating. However, the present disclosure is not limited thereto. The hydrophilic material layer 1 includes hydrophilic fibers, hydrophilic particles, or a combination thereof.

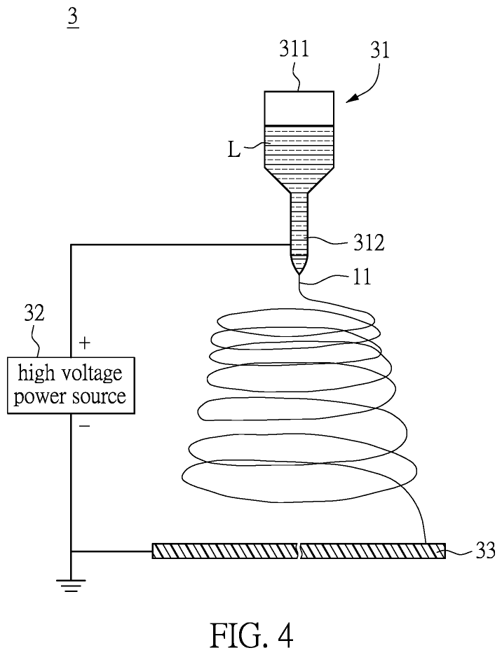

In some embodiments, the hydrophilic material layer 1 is formed by one or several hydrophilic fibers 11 (as shown in FIG. 3). The hydrophilic fibers 11 include a hydrophilic polymer as a material. The hydrophilic polymer is a macromolecule that is compatible with the human body. For example, the hydrophilic polymer can be hyaluronic acid, collagen, gelatin, alginate, chitosan, or a combination thereof. Further, a molecular weight of the hydrophilic polymer is from 2,000 to 2,000,000. Preferably, the molecular weight of the hydrophilic polymer is from 6,000 to 200,000.

In some embodiments, the hydrophilic fibers 11 can further include an excipient as a material. The excipient is provided for attachment of the hydrophilic polymer, so as to enhance structural characteristics of the hydrophilic material layer 1. The excipient can be, for example, polyvinyl alcohol (PVA), polyethylene glycol (PEG), polyvinylpyrrolidone (PVP), carboxymethyl cellulose, stearic acid, starch, or a combination thereof. In addition, a molecular weight of the excipient is from 1,000 to 2,000,000. Preferably, the molecular weight of the excipient is from 100,000 to 600,000. For example, when the excipient is polyvinyl alcohol, a molecular weight of the polyvinyl alcohol is less than or equal to 2,000,000. When the excipient is polyethylene glycol, a molecular weight of the polyethylene glycol is less than or equal to 1,000,000.

In other embodiments, the hydrophilic material layer 1 is formed by the multiple hydrophilic particles. Preferably, the hydrophilic particles have a particle size that ranges from 30 μm to 200 μm. The hydrophilic particles include a hydrophilic polymer as a material. The hydrophilic polymer is a macromolecule that is compatible with the human body. For example, the hydrophilic polymer can be hyaluronic acid, collagen, gelatin, alginate, chitosan, or a combination thereof. Further, a molecular weight of the hydrophilic polymer is from 2,000 to 2,000,000. Preferably, the molecular weight of the hydrophilic polymer is from 6,000 to 200,000.

In the present disclosure, a hydrophilic material layer including the hydrophilic fibers and a hydrophilic material layer including the hydrophilic particles are respectively formed by coating. Contact angles of these two hydrophilic material layers are measured, and measurement results are shown in Table 1. From the results of Table 1, it can be observed that the hydrophilic material layer including the hydrophilic particles has better hydrophilicity.

TABLE 1

| Hydrophilic material layer | Water contact angle |
|---|---|
| Hydrophilic material layer | 66 degrees to 78 degrees |
| including hydrophilic fibers | |
| Hydrophilic material layer | 59 degrees to 64 degrees |
| including hydrophilic particles | |

In some embodiments, the hydrophilic material layer 1 is jointly formed by hydrophilic fibers and hydrophilic particles. Materials of these hydrophilic fibers and hydrophilic particles are as previously described, and will not be reiterated herein.

When the hydrophilic material layer 1 is formed by the hydrophilic fibers 11, the hydrophilic material layer 1 can be formed by electrospinning or coating. In an exemplary embodiment, the hydrophilic material layer 1 is formed by electrospinning through use of an electrospinning device 3.

As shown in FIG. 4, the electrospinning device 3 mainly includes a spinneret 31, a high voltage power source 32, and a collection plate 33. The spinneret 31 includes a liquid storage tank 311 and a nozzle 312, and bottom portions of the nozzle 312 and the liquid storage tank 311 are in fluid communication with one another. A positive electrode and a negative electrode of the high voltage power source 32 are respectively and electrically connected to the nozzle 312 and the collection plate 33.

During use, an electrospinning solution L is first placed into the liquid storage tank 311. Then, through the high voltage power source 32, an electric field of a predetermined strength is generated between the spinneret 31 and the collection plate 33. After the electrospinning solution L is discharged out of the nozzle 312, the electrospinning solution L is cured to form the hydrophilic fibers 11 that are deposited on the collection plate 33. Through controlling a movement of the spinneret 31, the hydrophilic fibers 11 are tightly stacked, intertwined, or interwoven along a specific direction, so as to form the hydrophilic material layer 1 with a uniform thickness.

A solid content and a solvent are included as main components of the electrospinning solution L. The solvent is used to disperse the solid content. In the electrospinning solution L that is used to form the hydrophilic material layer, the solid content includes a hydrophilic polymer and an excipient. Further, the solvent is a water-based solvent that does not have cytotoxicity and satisfies medical device requirements. For example, the solvent can be a mixture of water and alcohol, in which a weight ratio between the water and the alcohol is from 1:9 to 9:1.

In an exemplary embodiment, the electrospinning solution L that is used to form the hydrophilic material layer 1 includes 5 wt % to 75 wt % of the alcohol, 25 wt % to 95 wt % of the water, 0.5 wt % to 5 wt % of the hydrophilic polymer, and 2 wt % to 15 wt % of the excipient. However, the present disclosure is not limited thereto.

Furthermore, adjustable parameters of the electrospinning device 3 include: a concentration of the electrospinning solution L, a spinning temperature, a strength of the electric field, a collection distance (otherwise referred to as a deposition distance), a collection time, etc. In the present embodiment, the spinning temperature is from 5° C. to 95° C., and is preferably from 10° C. to 90° C. A voltage strength is from 5 kV to 60 kV, and is preferably from 10 kV to 25 kV. A discharge speed of the electrospinning solution L is from 0.1 cc/min to 10 cc/min, and the collection distance between the nozzle 312 and the collection plate 33 is from 15 cm to 90 cm. However, these details only describe possible implementations of the electrospinning, and should not be taken as limiting the scope of the present disclosure.

The material of the hydrophilic fibers 11 includes the above-mentioned hydrophilic polymer and excipient (i.e., the solid content in the electrospinning solution L). A weight ratio between the hydrophilic polymer and the excipient is from 1:30 to 5:1. Preferably, the weight ratio between the hydrophilic polymer and the excipient is from 1:10 to 3:1.

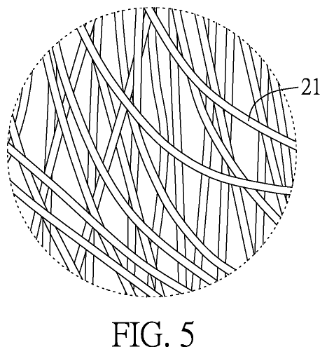

As shown in FIG. 5, the hydrophobic material layer 2 of the present disclosure can also be formed by one or several hydrophobic fibers 21. However, the present disclosure is not limited thereto. When examined microscopically, the hydrophobic material layer 2 is a porous structure. The hydrophobic fibers 21 have a diameter that is from 200 nm to 3,000 nm, so that the hydrophobic material layer 2 has a proper structural strength for serving as a substrate. However, the structure of the hydrophobic material layer 2 is not limited thereto. The hydrophobic material layer 2 can also be a solid layer or a layer having other structures.

The hydrophobic material layer 2 can be formed by electrospinning, coating or extrusion. In an exemplary embodiment, the hydrophobic material layer 2 is formed by electrospinning, and its formation process is similar to that which is described above. The difference resides in that an electrospinning solution L for forming the hydrophobic material layer 2 and the electrospinning solution L for forming the hydrophilic material layer 1 are not the same.

The electrospinning solution L for forming the hydrophobic material layer 2 includes a solid content and a solvent, and the solid content includes a hydrophobic polymer.

The hydrophobic material layer 2 includes the hydrophobic polymer as a material. The hydrophobic polymer can be polylactic acid (PLA), polycaprolactone (PCL), poly(lactic-co-glycolic acid) (PLGA), polyhydroxyalkanoates (PHA), polyglycolic acid (PGA), or a combination thereof.

In an exemplary embodiment, the hydrophobic polymer is polylactic acid. The hydrophobic polymer includes both poly-D-lactic acid and poly-L-lactic acid. It should be noted that when the hydrophobic polymer is the polylactic acid, the electrospinning solution L must include both the poly-D-lactic acid and the poly-L-lactic acid; otherwise, the polylactic acid cannot form into a uniform dispersion liquid. In some embodiments, a weight ratio between the poly-D-lactic acid and the poly-L-lactic acid is from 1:9 to 9:1. Preferably, the weight ratio between the poly-D-lactic acid and the poly-L-lactic acid is from 5:5 to 8:2. More preferably, a content of the poly-D-lactic acid is greater than that of the poly-L-lactic acid.

The solvent is an organic solvent that does not have cytotoxicity. For example, the solvent can be one of acetone, butanone, ethylene glycol, hexafluoroisopropanol (HFIP), and isopropanol and one of deacetylated chitin (DAC), N,N-dimethylformamide (DMF), dimethylacetamide (DMAC), dimethyl sulfoxide (DMSO), and diethyl ether. In an exemplary embodiment, the organic solvent is a mixture of the acetone and the dimethylacetamide. In addition, a weight ratio between the acetone and the dimethylacetamide is from 1:9 to 9:1.

In an exemplary embodiment, the electrospinning solution L for forming the hydrophobic material layer 2 includes 99 wt % of the butanone and 0.5 wt % to 15 wt % of the hydrophobic polymer. However, the present disclosure is not limited thereto.

It is worth mentioning that crystallinity of the hydrophobic polymer will not be affected when hydrophobic fibers are formed by electrospinning Therefore, compared with a hydrophobic material layer 2 formed by hot pressing, the hydrophobic material layer 2 that is formed by electrospinning in the present disclosure has a better tensile strength.

In an exemplary embodiment, both of the hydrophilic material layer 1 and the hydrophobic material layer 2 are formed by electrospinning. When examined microscopically, there is a transitional area between the hydrophilic material layer 1 and the hydrophobic material layer 2. In the transitional area, the hydrophilic fibers 11 and the hydrophobic fibers 22 are interwoven with each other along a thickness direction, so that the hydrophilic material layer 1 and the hydrophobic material layer 2 have a sufficient bonding strength therebetween. Accordingly, the double-layered medical membrane of the present disclosure has good elasticity and extension properties.

A single-layered anti-adhesion membrane that is currently available on the market is formed by polylactic acid and by way of extrusion. According to the test standard of ASTM D882, a tensile strength of such an anti-adhesion membrane is 0.3 MPa. On the other hand, the double-layered medical membrane of the present disclosure includes the hydrophilic material layer 1 and the hydrophobic material layer 2 that are respectively formed by way of electrospinning, According to the test standard of ASTM D882, a tensile strength of the double-layered medical membrane is 2.58 MPa.

A method for manufacturing the double-layered medical membrane of the present disclosure includes the following steps: forming the hydrophilic material layer; and producing the double-layered medical membrane that includes the hydrophilic material layer. The hydrophilic material layer includes the hydrophilic fibers, the hydrophilic particles, or the combination thereof. The hydrophilic fibers or the hydrophilic particles include the hydrophilic polymer as a material. The double-layered medical membrane further includes the hydrophobic material layer that is disposed on the hydrophilic material layer.

It should be noted that there is no specific sequence for forming the hydrophilic material layer 1 and the hydrophobic material layer 2. That is to say, the hydrophobic material layer 2 can first be formed, and then the hydrophilic material layer 1 is formed on the hydrophobic material layer 2. Alternatively, the hydrophilic material layer 1 can first be formed, and then the hydrophobic material layer 2 is formed on the hydrophilic material layer 1.

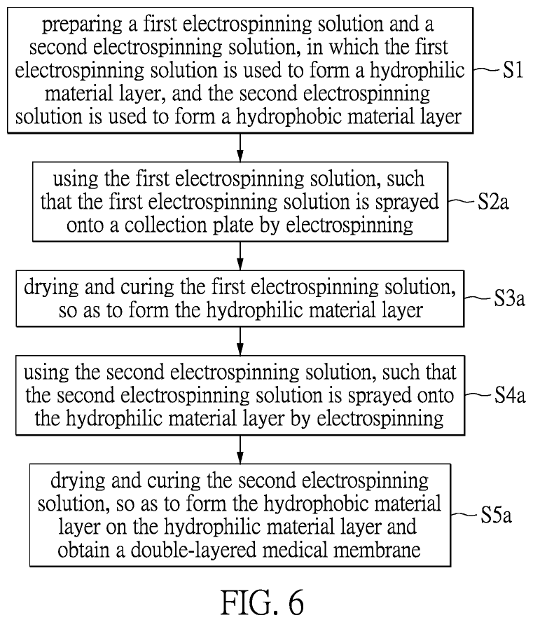

Reference is made to FIG. 6. In one embodiment of the present disclosure, the method for manufacturing the double-layered medical membrane includes steps as follows. Step S1: preparing a first electrospinning solution and a second electrospinning solution, in which the first electrospinning solution is used to form the hydrophilic material layer 1, and the second electrospinning solution is used to form the hydrophobic material layer 2. Step S2a: using the first electrospinning solution, such that the first electrospinning solution is sprayed onto the collection plate 33 by electrospinning Step S3a: drying and curing the first electrospinning solution, so as to form the hydrophilic material layer 1. Step S4a: using the second electrospinning solution, such that the second electrospinning solution is sprayed onto the hydrophilic material layer 1 by electrospinning Step S5a: drying and curing the second electrospinning solution, so as to form the hydrophobic material layer 2 on the hydrophilic material layer 1 and obtain the double-layered medical membrane.

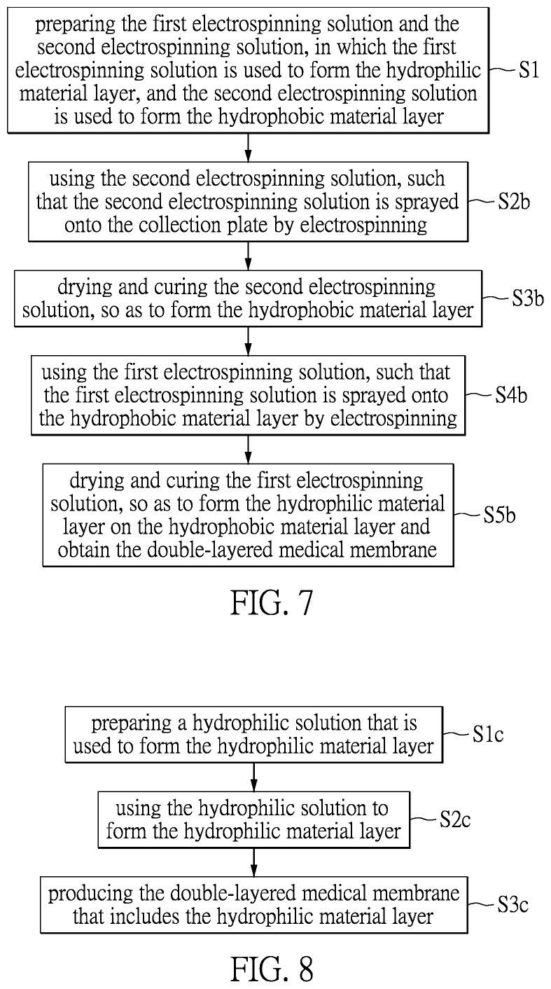

Reference is made to FIG. 7. In another embodiment of the present disclosure, the method for manufacturing the double-layered medical membrane includes steps as follows. Step S1: preparing the first electrospinning solution and the second electrospinning solution, in which the first electrospinning solution is used to form the hydrophilic material layer 1, and the second electrospinning solution is used to form the hydrophobic material layer 2. Step S2b: using the second electrospinning solution, such that the second electrospinning solution is sprayed onto the collection plate 33 by electrospinning Step S3b: drying and curing the second electrospinning solution, so as to form the hydrophobic material layer 2. Step S4b: using the first electrospinning solution, such that the first electrospinning solution is sprayed onto the hydrophobic material layer 2 by electrospinning Step S5b: drying and curing the first electrospinning solution, so as to form the hydrophilic material layer 1 on the hydrophobic material layer 2 and obtain the double-layered medical membrane.

The double-layered medical membrane manufactured by the above-mentioned method has good elasticity and extension properties. In addition, the transitional area is provided between the hydrophilic material layer 1 and the hydrophobic material layer 2. In the transitional area, the hydrophilic fibers 11 and the hydrophobic fibers 22 are interwoven with each other along the thickness direction, so that the hydrophilic material layer 1 and the hydrophobic material layer 2 have a sufficient bonding strength therebetween.

Reference is made to FIG. 8. In yet another embodiment of the present disclosure, the method for manufacturing the double-layered medical membrane includes steps as follows. Step S1c: preparing a hydrophilic solution that is used to form the hydrophilic material layer 1. The hydrophilic material layer 1 includes the hydrophilic fibers, the hydrophilic particles, or the combination thereof, and the hydrophilic fibers and the hydrophilic particles include the hydrophilic polymer as a material. Step S2c: using the hydrophilic solution to form the hydrophilic material layer 1. The hydrophilic material layer 1 can be formed by coating (but not limited thereto). Step S3c: producing the double-layered medical membrane that includes the hydrophilic material layer 1. In the step S3c, the hydrophilic material layer 1 can be formed on the hydrophobic material layer 2. Or, the hydrophilic material layer 1 can first be formed, and then the hydrophobic material layer 2 is disposed on the hydrophilic material layer 1.

### Beneficial Effects of the Embodiments

In conclusion, in the double-layered medical membrane and the method for manufacturing the same provided by the present disclosure, by virtue of “the hydrophilic material layer 1 being formed by the hydrophilic fibers 11, the hydrophilic particles, or the combination thereof” and “the hydrophobic material layer 2 being disposed on the hydrophilic material layer 1,” inconveniences associated with using a conventional single-layered medical membrane can be solved.

More specifically, by virtue of “the hydrophilic material layer 1 being formed by electrospinning,” the tensile strength of the double-layered medical membrane can be enhanced.

More specifically, by virtue of “the hydrophilic material layer 1 including the hydrophilic fibers,” hydrophilic properties of the hydrophilic material layer 1 in the double-layered medical membrane can be enhanced.

More specifically, by virtue of “the hydrophobic material layer 2 being formed by the hydrophobic fibers 21, and the hydrophobic fibers 21 having a diameter that is from 200 nm to 3,000 nm,” a structural strength and the tensile strength of the double-layered medical membrane can be enhanced.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A double-layered medical membrane, comprising:
a hydrophilic material layer, wherein the hydrophilic material layer includes hydrophilic fibers, hydrophilic particles, or a combination thereof;
wherein the hydrophilic fibers and the hydrophilic particles include a hydrophilic polymer as a material; and
a hydrophobic material layer disposed on the hydrophilic material layer.

2. The double-layered medical membrane according to claim 1, wherein the hydrophilic polymer is selected from the group consisting of hyaluronic acid, carboxymethyl cellulose, collagen, gelatin, alginate, and chitosan.

3. The double-layered medical membrane according to claim 1, wherein a molecular weight of the hydrophilic polymer is from 2,000 to 2,000,000.

4. The double-layered medical membrane according to claim 1, wherein the hydrophilic fibers further include an excipient as a material, and a weight ratio between the hydrophilic polymer of the hydrophilic fibers and the excipient is from 1:30 to 5:1.

5. The double-layered medical membrane according to claim 1, wherein the hydrophobic material layer is formed by hydrophobic fibers, and the hydrophobic fibers have a diameter that is from 200 nm to 3,000 nm.

6. The double-layered medical membrane according to claim 1, wherein the hydrophobic material layer is formed by electrospinning.

7. The double-layered medical membrane according to claim 1, wherein a tensile strength of the double-layered medical membrane is from 1.1 MPa to 5.2 Mpa.

8. A method for manufacturing a double-layered medical membrane, comprising:
using an electrospinning solution to form a hydrophilic material layer by electrospinning, wherein the hydrophilic material layer includes hydrophilic fibers, and the hydrophilic fibers include a hydrophilic polymer as a material; and
producing the double-layered medical membrane that includes the hydrophilic material layer, wherein the double-layered medical membrane further includes a hydrophobic material layer that is disposed on the hydrophilic material layer.

9. The method according to claim 8, wherein the hydrophilic fibers further include an excipient as a material; wherein the electrospinning solution includes: 5 wt % to 75 wt % of alcohol, 25 wt % to 95 wt % of water, 0.5 wt % to 5 wt % of the hydrophilic polymer, and 2 wt % to 15 wt % of the excipient.

10. A method for manufacturing a double-layered medical membrane, comprising:
using a hydrophilic solution to form a hydrophilic material layer, wherein the hydrophilic material layer includes hydrophilic fibers, hydrophilic particles, or a combination thereof; wherein the hydrophilic fibers and the hydrophilic particles include a hydrophilic polymer as a material; and
producing the double-layered medical membrane that includes the hydrophilic material layer, wherein the double-layered medical membrane further includes a hydrophobic material layer that is disposed on the hydrophilic material layer.
